(19) United States
(12) Patent Application Publication
(10) Pub. No.: US 2016/0331990 A1
(43) Pub. Date: Nov. 17, 2016

(54) UNI-POLAR ROTATING ELECTROMAGNETIC MEDICAL APPARATUS AND METHODS OF USE

(71) Applicants: Hossam Abdel Salam El Sayed MOHAMED, Ottawa (CA); Houda Abdul Rahman M. Al MANSOUR, Ottawa (CA)

(72) Inventors: Hossam Abdel Salam El Sayed MOHAMED, Ottawa (CA); Houda Abdul Rahman M. Al MANSOUR, Ottawa (CA)

(21) Appl. No.: 15/111,334

(22) PCT Filed: Jan. 15, 2014
(86) PCT No.: PCT/IB2014/000047

§ 371 Date: Jul. 13, 2016

(30) Foreign Application Priority Data
Feb. 6, 2014 (IL) 231186

(51) Int. Cl. A61N 2/12; A61N 2/06; A61N 2/00

(52) U.S. Cl. CPC A61N 2/12; A61N 2/006; A61N 2/008; A61N 2/004; A61N 2/06

(57) ABSTRACT

Apparatus and methods for providing uni-polar pulsed magnetic field therapies for various medical conditions are disclosed. The apparatus produces the pulsed uni-polar magnetic field by repeatedly moving at least one very strong permanent magnet through a prescribed path (e.g., a circular path) over an anatomic region of interest in the patient. The apparatus includes a housing in which the at least one magnet is located and a body shielding material, e.g., a Mu-metal shield, to shape the field to make it of fectively uni-polar. The movement of the magnet through the path is accomplished by means of a motor.

### CROSS-REFERENCE TO RELATED APPLICATIONS

N/A

### FIELD OF THE INVENTION

This invention relates generally to medical devices and methods of use and more particularly to Pulsed Electromagnetic Field (“PEMF”) apparatus making use of at least one movable permanent magnet and methods of use for treating various medical conditions.

### BACKGROUND OF THE INVENTION

The therapeutic application of Pulsed Electromagnetic Field (“PEMF”) therapy has been accepted by the Food and Drug Administration for use in humans and has become an accepted modality in treating various medical conditions. In particular, it is now used in treatment of orthopedic diseases like osteoporosis, muscle pain, arthritis, synovitis, tendinitis, neck pain, back pain and others. It is also used in treatment of aging processes of the brain, such as Alzheimer's disease, Parkinson's disease, dementia. It has also been used to treat coronary artery disease too. One of the most successful usages is for the treatments of fibro-myalgia. As is known this disease is characterized by muscle pain, sleep disturbance, generalized weakness, with some psychological elements. Typically, it has a poor response to medical treatments, except for PEMF therapy.

Typically PEMF therapy is achieved by means of apparatus making use of at least one inductive coil producing a pulsating electromagnetic field. The coil(s) is/are energized by applying a predetermined electrical current to one or more of the inductive coil(s) in order to produce a desirable magnetic field with specified field characteristics.

In our copending PCT Application PCT/IB2013/002441, entitled Uni-Polar Pulsed Electromagnetic Medical Apparatus And Methods, filed on Oct. 30, 2013, whose disclosure is incorporated by reference herein, there is disclosed apparatus and methods which overcome many of the disadvantages of the prior art PEMF apparatus. In particular, the apparatus and methods of that application provide an effectively uni-polar electromagnetic field in a focused or concentrated path directed to the exact pathological site so that it can be treated with high accuracy and with optimum dose whatever the depth of the region of interest. That apparatus and it methods of use, entails use of an electromagnet to generate the field. The strength of the magnetic field produced by that apparatus is related to the number of loops of conductors forming the electromagnet and the amount of electrical current utilized. Therefore, the magnetic energy produced from that apparatus can be carefully controlled. However, because the electrical energy passing through the coiled conductors making up the electromagnet of that apparatus, as well as the electromagnetics of other prior art PEMF apparatus, will heat up those conductors, such action precludes the use of the PEMF apparatus for long periods of time. Thus, heretofore PEMF instruments are typically operated on a repeated shut-down basis, e.g., the instruments are repeatedly shut down to cool at approximately 20 minute or so intervals.

A need thus exists for PEMF apparatus which overcomes that drawback of the prior art to provide a pulsed electromagnetic field with high energy that can penetrate into deeply located tissues over a relatively long period of time without the production of heat.

The subject invention addresses that need. In particular, as will be seen from the discussion to follow the subject invention makes use of at least one movable permanent magnet in lieu of an electromagnet to create the PEMF. To achieve the pulsing action, the at least one permanent magnet is periodically moved in a path, e.g., rotated in a circular path, across the anatomic region of the patient to be treated (hereinafter referred to as the “region of interest”) to effectively apply uni-polar magnetic energy thereto.

### SUMMARY OF THE INVENTION

In accordance with one aspect of the invention there is provided apparatus for applying uni-polar magnetic energy to an anatomic region of interest located within the body of a patient. The apparatus basically comprises at least one permanent magnet, a body of Mu-metal shielding, and a motor. The at least one permanent magnet produces a magnetic field having a longitudinal axis, a first pole of a first polarity and a second pole of a second polarity, with the longitudinal axis extending through the poles and with lines of flux of the magnetic field extending outward from the first pole generally parallel to the longitudinal axis. The at least one permanent magnet is surrounded by the body of Mu-metal shielding except for the first pole of the at least one permanent magnet so that the first pole is exposed and from which lines of flux of the magnetic field emanate. The at least one magnet is coupled to the motor, which is arranged to be operated to repeatedly move the at least one magnet through a predetermined path, whereupon a portion of that path can be disposed adjacent the anatomic region of interest of the patient to cause the exposed pole of the at least one magnet to periodically direct its lines of flux to the region of interest in the patient.

Other aspects of this invention entail various methods of treating medical conditions of a patient by applying a pulsed uni-polar magnetic field to a region of interest in the body of that patient. Those methods basically comprise providing apparatus similar to that described above, disposing the apparatus adjacent the region of interest of the patient and operating the motor to cause the at least one magnet to periodically traverse a path to direct the lines of flux produced by the first pole of the at least one magnet to the region of interest in the body of the patient.

### DESCRIPTION OF THE DRAWING

FIG. 1 is a cross-sectional view of one exemplary embodiment of apparatus for providing a uni-polar PEMF therapy constructed in accordance with this invention, that apparatus including a pair of rotating permanent magnets;

FIG. 2 is an isometric illustration of a portion of the apparatus shown in FIG. 1 to show the rotary path that the magnets of the apparatus take to periodically bring them over a region of interest in the body of a patient to treat that region of interest; and

FIG. 3 is a cross-sectional view similar to FIG. 1 but showing an alternative exemplary embodiment of the apparatus of this invention.

### DETAILED DESCRIPTION OF THE PREFERRED EMBODIMENT

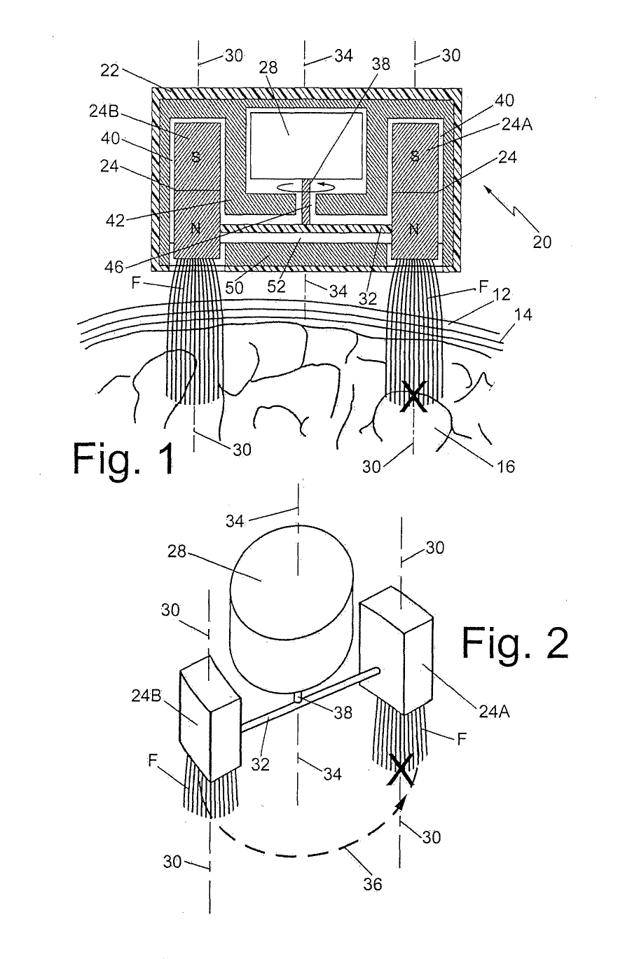

Referring now to the various figures of the drawing wherein like reference characters refer to like parts, there is shown in FIG. 1 at 20 apparatus for producing a uni-polar pulsed magnetic field which can be directed to great depths into the body of a patient to provide therapy to an interior anatomic region of interest.

The apparatus 20 basically comprises a housing 22, at least one permanent magnet 24, Mu-metal shielding 26, and a motor 28. The at least one permanent magnet is arranged to be periodically moved in a path across a region of interest in the body of the patient to provide uni-polar pulsed magnetic therapy to that region. In the exemplary embodiments of the invention shown in the drawing the at least one permanent magnet is in the form of two permanent magnets 24A and 24B. Those magnets are of identical construction and each preferably produces a very strong magnetic field. In particular, in one exemplary embodiment each of the. magnets 24A and 24B is formed of super-magnet material, e.g., Neodymium N52. Other super-magnetic materials can be used as well. Preferably the magnetic field produced by the magnets has a magnetic flux density in the range of 14,000 to 15,000 gauss and a magnetic field strength in the range of 14,000˜15,000 oersteds.

Each magnet is an elongated member that has a central longitudinal axis 30 extending through it, a North pole (designated by the letter N) and a South pole (designated by the letter S). The magnetic field F produced by each magnet is represented by the lines of flux emanating from its pole. The magnetic field F is centered about the longitudinal axis 30. The exemplary apparatus 20 is arranged to produce a uni-polar field emanating from the North pole of its magnets for reasons to be described later, with each field being centered about the longitudinal axis of the magnet from which it emanates. The two magnets 24A and 24B are mounted on respective, opposite ends of an elongated bridge member 32. The bridge member 32 is arranged to be rotated about a rotation axis 34 to carry the North poles of the two magnets 24A and 24B through a repeating circular path 36 (FIG. 2) by the operation of the motor 28. This action repeatedly brings the lines of flux from each magnet 24A and 24B over the region of interest of the patient that lies under a portion of the circular path 36. In FIG. 1 the region of interest is designated by an. X. As will be appreciated from the discussion to follow during each revolution of the bridge member, each magnet 24A and 24B will be momentarily aligned with the region of interest X as the magnet traverses the path 36 to thereby periodically expose the region of interest to the magnetic field. Hence the region of interest receives what is effectively a pulsed magnetic field.

The motor 28 can be any suitable type, e.g., an electric motor, having a rotating output or drive shaft 38. The motor can be located within the housing 22, as shown, with its rotary drive shaft 38 connected to the midpoint of the bridge member 32 on the rotation axis 34. Alternatively, the motor 28 may be located outside the housing 22. In that case the rotary output shaft 38 of the motor 28 is coupled to the bridge member 32 by a cable or some other flexible or rigid drive member. Control of the motor can be accomplished by any suitable means, e.g., a microprocessor, or any other suitable circuitry (not shown).

The magnetic field F is shaped and collimated by the shielding 26 so that the lines of flux extending outward from the North pole of each magnet are generally parallel to the longitudinal axis of the magnet. In particular, the Mu-metal shielding 26 comprises a body located within the housing 22 and having an annular channel or slot 40 through which the magnets 24A and 24B move when the motor is operated. The shielding body 26 includes a central portion 42 including a cavity 44 in which the motor 28 is located and a central opening 46 through which the motor's drive shaft 38 extends. The Mu-metal body 26 also includes bottom wall section 50 spaced from the central portion 42 to form a disk-like cavity 52 in communication with the annular channel 40 through which the bridge member 32 may rotate to carry the magnets through their circular path 36 over the region of interest X. The annular channel 40 is open adjacent the bottom wall section 50 to expose the North poles of the magnets 24A and 24B, whereas the body of the Mu-metal shielding completely surrounds the rest of each of those magnets. Accordingly, the lines of flux of the field produced by each magnet is concentrated or collimated by the surrounding Mu-metal shield so that it extends generally parallel to the central longitudinal axis 30 of each magnet for a substantial distance beyond the end of the housing of the unit as shown in FIG. 1 to apply the pulsed magnetic energy to the region of interest.

It should be noted that while the lines of flux produced by each magnet do curve back to the magnet's South pole, they are shaped and confined by the Mu-metal shield so that their effect on the region of interest X is minimal, if any. Thus the shielding 26 results in the production of a magnetic field emanating from the apparatus 20 which is effectively uni-polar. In particular, the portion of the field which emanates from the North pole end of the housing 22 is shaped so that it is condensed or collimated. This collimated uni-polar EM field can be directed into the patient's body to effectively reach any region of interest to be treated irrespective of the depth of that region of interest.

As mentioned above, in the exemplary embodiment shown in FIG. 1, the uni-polar magnetic field is of North polarity, i.e., it consists of the concentrated lines of flux from the North pole of each magnet, since for many therapies it is desired to utilize the North pole as the treating modality, inasmuch as the North pole seems to provide better physiological effects on human cells. Thus, in the exemplary embodiment the apparatus 20 is arranged to have the North poles of the magnets 24A and 24B exposed, with the South poles of those magnets being shielded by the Mu metal body 26. For other applications, the South pole of the apparatus 20 may be utilized. In such a case the South poles of the magnets will be exposed and with the Mu metal body 26 surrounding the North poles of those magnets.

Since the magnets 24A and 24B will be periodically rotated over the region of interest X, the uni-polar magnetic energy provided by the apparatus will be pulsed, i.e., there will be a “on” time period during each complete revolution of the bridge member, wherein the magnetic flux emanating from the magnet 24A is travelling across the region of interest X, thereby applying magnetic energy to that region, and an “off” time period during that revolution when the magnet 24A will be in another portion of the path 36 (i.e., not over the region of interest X) and hence not directing the magnetic flux to the region of interest. So too, there will be an “on” time period during each complete revolution of the bridge member, wherein the magnetic flux emanating from the magnet 24B is travelling across the region of interest X, thereby applying magnetic energy to that region, and an “off” time period during that revolution when the magnet 24B will be in another portion of the path 36 (i.e., not over the region of interest X) and hence not directing the magnetic flux to the region of interest.

As will be appreciated by those skilled in the art the rotational speed of the bridge member, the number and the physical size of the magnets will all be factors in the “on” and “off” time periods of the duty cycle of operation of the apparatus 20 (i.e., the time that the region of interest X is exposed to the collimated magnetic field). In accordance with a preferred exemplary embodiment of this invention the ratio of the on-to-off periods of time is preferably in the range of 30%-40% on and 70%-60% off, at an on-off pulse rate of approximately 25 to 50 per second. If one super-magnet is utilized, the rate of the pulse would be 25 per second, but if 2 magnets are used, the rate of the pulse would be 50 per second. Normally, the natural pulsation from the earth is 30 Hz. Recently, it has been discovered that healing effect is directly proportional to the increase in the pulse rate up to 50 Hz, then the healing effect gradually becomes inversely proportional after 50 Hz, e.g., a duration of 8 milliseconds “on: and about 12 milliseconds is “off”. That duty cycle, is merely exemplary and other duty cycles can be utilized depending upon conditions. In any case, the duty cycle can be repeated for as long as desired, since the apparatus will not become unduly heated, as is the case of prior art devices making use of electromagnets.

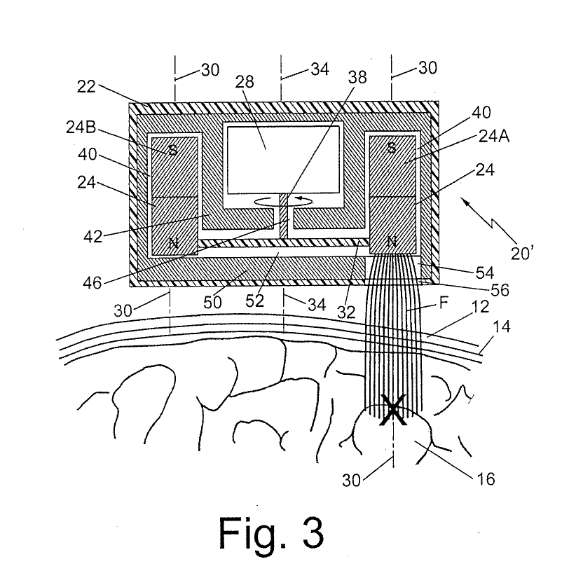

In FIG. 3 there is shown an alternative embodiment of an apparatus 20′ constructed in accordance with this invention. The apparatus 20′ is similar to the apparatus 20 shown in FIG. 1, except that it is arranged to enable the magnetic field to exit the apparatus at only a predetermined location, e.g., at a “window” (to be described shortly) along the circular path 36 through which the magnets 24A and 24B are carried, whereas the magnetic field produced by each of the magnets of the embodiment 20 exits the apparatus along the entire length of the circular path. Thus, with the embodiment 20 of FIG. 1, the apparatus should be oriented and juxtaposed with respect to the region of interest X to be treated so that only a portion of the path of those magnets is aligned with the region of interest to result in the application of a pulsed uni-polar magnetic field thereto. In contradistinction, since the apparatus 20′ only allows the magnetic field to exit the apparatus at the location of the window, all that is required in the use of that apparatus is to orient and dispose the apparatus 20′ so that its window is aligned with the region of interest X.

Inasmuch as the apparatus 20′ is similar in construction to the apparatus 20, the common components of those apparatus and their manner of operation will not be reiterated in the interest of brevity. Moreover, those common components will be given the same reference numbers. Thus, as can be seen in FIG. 3 the shielding body 36 includes a window or opening 54 which forms a short segment of the annular channel 40. The remainder of the bottom wall 50 of the shielding body 26 is solid. The housing 22 is constructed so that it includes a window or opening 56 which overlies the window 54 in the Mu-metal shield.

Use of the apparatus of this invention, like the exemplary apparatus 20 or 20′ (or any other apparatus constructed in accordance with this invention) for providing various therapies in accordance with this invention will now be described To that end, FIG. 1 is an illustration of the exemplary apparatus 20 of FIG. 1 shown in the process of treating a brain disorder or condition, such as but not limited to some brain pathology selected from the group comprising Alzheimer's disease, Parkinson's disease, dementia, migraine and senile atrophy of the brain. As can be seen, the apparatus 20 is disposed adjacent the skin 12 of the patient 10 at a desired position adjacent the patient's skull 14 and juxtaposed and oriented so that a portion of the annular channel 40 forming the path 36 is aligned with the region of interest X to direct the pulsed uni-polar magnetic field to region of interest X in the brain 16 to treat the particular pathology. When the apparatus 20 is in the desired position and orientation the motor 28 may be activated (turned on). This can be accomplished by pressing an ON/OFF switch or button (not shown) on the apparatus' housing cause the bridge member to carry the magnets 24A and 24B in the circular path 36 across the region of interest. Thus, the magnetic field produced by each magnet is directed along its longitudinal central axis 30 to impinge the region of interest X each time that a magnet is disposed thereover. Since the uni-polar pulsed magnetic field is concentrated, focused and centered on the axis 30 it can thus penetrate through the skin and underlying tissue and bone of the skull to the situs of the pathology. The apparatus 20′ is used in a somewhat similar manner. In particular, the apparatus 20′ is juxtaposed and oriented with respect to the patient's head so that the windows 54 and 56 are aligned with the region of interest X. The apparatus 20 may then be operated to provide the uni-polar pulsed magnetic field to that region of interest.

As is well known, many aging brain diseases or conditions are caused by damage (degenerative changes) that occurs in a specific site in brain substance which will lead to a decreased local energy production in this area, with subsequent reduction in electro-chemical transmission. Thus, the higher amount of energy carried by the magnetic field into the region of interest without significant attenuation by the intervening tissue/bone should bring, better results and accelerate the healing process as compared to the prior art so it can be used as an important core therapy, not only as an adjuvant therapy.

The apparatus of this invention can also be used for treating chronic coronary diseases, such as coronary artery disease (CAD), cardio-myopathy, a previous myocardial infarction (death and fibrosis of part of the heart muscle) in those patients who have a high incidence of repeated infarction. As is known a blockage or even narrowing of certain blood vessels to certain part(s) of cardiac muscle will decrease oxygen and nutrition to that area with subsequent a decrease of local energy production. Thus, the affected area becomes weak and incapable of performing properly. The exposure of that cardiac tissue to the pulsed uni-polar magnetic field provided by the apparatus of this invention should compensate for that lack of the energy production, by providing additional energy to the cardiac tissue resulting from the collision of the pulsed uni-polar magnetic field with that cardiac tissue. The apparatus of the subject invention is particularly suitable for this type of application since the heart is a deep seated structure lying behind the skin and subcutaneous tissues, chest wall muscles and ribs. In obese persons (a quite common type of cardiac patient) location of the region of interest in the heart is likely to be even more remote since such patients have excess fat intervening between the skin and the region of interest. Thus, a very small amount of energy reaches to the heart by the techniques of the prior art. The technique of the subject invention, like the technique of our copending PCT Application PCT/IB2013/002441, Moreover, there is a superiority of the subject invention over those prior art techniques, namely, the ability of this invention to be used for extended periods of time, e.g., many hours, without a need to shut it down. This feature enables the application of a very large amount of energy via its cumulative effect. Thus, to treat a cardiac condition the apparatus of this invention is disposed adjacent the skin of the patient at any desired position on the patient's torso and operated as discussed above to direct the uni-polar pulsed magnetic field to the heart at the situs of the region to be treated.

The apparatus of this invention can also be used for providing orthopedic therapy, e.g., to direct the pulsed uni-polar magnetic field to a bone of a patient to treat osteoporosis thereat. To that end, the apparatus of this invention is disposed adjacent the skin of the patient and oriented to direct the uni-polar pulsed magnetic field to the situs of the osteoporosis. When the apparatus is in the desired and orientation position the ON/OFF button can be pressed to turn the apparatus on and thereby produce the uni-polar pulsed magnetic field. Since that field is concentrated and focused it can thus penetrate through the skin, subcutaneous tissue and intervening thigh muscles to the region of interest at the situs of the osteoporosis to reverse or at least impede the progress of the osteoporosis thereat.

The apparatus of this invention can also be used to treat avascular necrosis (AVN). AVN is bone death and commonly results from a chronic deficiency of blood supply to certain part of bone. Lack of blood supply to certain part of bone reduces the maintenance of bone turnover with subsequent degenerative changes in that area. The accumulative effect of the lack of an adequate blood supply to certain bone may end by death of that bone because of lack of local energy production. PEMF offers a good response in the treatment of this condition because collision of the pulsed electromagnetic field and that bone generate energy that can compensate for the lack of energy resulting from an insufficient blood supply. Thus, PEMF offers a modality which may avoid the necessity of surgical intervention to treat the AVN. However, since the affected bone is usually in a deep seated position because it is supplied by end organ artery. e.g., the head of the femur and the inner femoral condyle of knee, it is difficult to treat with prior art PEMF apparatus. The apparatus of subject invention, with its ability to reach deep seated structures provides an excellent means to treat AVN. To that end, the apparatus of this invention can be used in a similar manner to that described above to deliver the concentrated pulsed uni-polar magnetic field over long period of time without a need to shut it down to the situs of the avascular necrosis.

The apparatus of this invention can also be utilized to treat a non-union fracture or a delayed healing after a fracture. That can be accomplished by disposing the apparatus adjacent the skin of the patient and orienting it to direct the uni-polar pulsed magnetic field to the situs of the fracture. When the apparatus is in the desired position and orientation the ON/OFF button can be pressed to turn the apparatus on and thereby produce the pulsed uni-polar magnetic field. Since that field is concentrated and focused it can thus penetrate through the skin and intervening thigh muscles to the region surrounding the fracture, e.g., the region of interest along the bone portions contiguous with the fracture to facilitate its healing. The application of a pulsed uni-polar magnetic field to treat bone fracture with delayed union and non-union should provide promising results by accelerating the healing process making use of the energy of that magnetic field to activate stem cells that enhance new bone formation. Thus, the apparatus of this invention can be used as an important core therapy, not only as an adjuvant therapy.

The apparatus of this invention can also be used to treat osteomyelitis. Chronic osteomyelitis (septic bone infection) is catastrophic disease that is very difficult to treat and a full cure is sometimes impossible. It needs a long term course of treatment, sometimes over years, with repeated surgical intervention in many cases. With this disease, bacteria live in a devitalized bone and hence are remote from the immune system. PEMF should prove beneficial for this condition by producing local energy at the situs of the infection. This energy, in turn, should increase the blood supply and raise the patient's immunity by increasing the number of immune cells (macrophages and lymphocytes) in the affected devitalized bone. Thus, to treat osteomyelitis the apparatus of this invention can be brought into position closely adjacent and oriented toward the situs of an osteomyelitis infection and then operated in a similar manner to that described above to deliver the concentrated pulsed uni-polar magnetic field to the situs of the infection.

The apparatus of this invention can also be used to treat chronic low back pain by directing the pulsed uni-polar magnetic field to the facet joints, the back of the vertebral bodies and intervening discs of the spine. Chronic low back pain is a group of diseases that affects a large number of the population. One common cause of low back pain is that the intervertebral disc herniates and compresses the spinal nerve causing severe neurological pain. In case of the neck, the pain is referred to shoulders. In case of lumbar spine the pain is radiated or referred to the legs. The mechanism of pain is compression of the spinal nerve by the protruded substance of the disc with subsequent reduction of blood supply of affected nerve resulting in reduction of local energy production in the affected nerves. The treatment with PEMF directly supplies the energy to these compressed nerves and thus can reverse the whole pathological process by supplying the local tissues with a free energy. Therefore, the pain can gradually subside. Thus, to treat low back pain the apparatus of this invention can be brought into position closely adjacent and oriented toward the region of interest and then operated in a similar manner to that described above to deliver the concentrated pulsed uni-polar magnetic field to the region of interest.

Joint arthritis is another disease that can be treated by the apparatus of this invention. Osteoarthritic joint diseases are degenerative joint diseases which break down the articular cartilage which become thin, eroded, and sometimes ulcerated with reduction of lubricant materials (e.g., synovial fluids) leading to painful movements. The result of this condition typically involves protective reduction of the movements of the patient with subsequent muscle weakness. Consequently, there will be joint instability resulting in further damage of articular cartilage and the patient enters in a vicious cycle. Treatment with PEMF offers a way to improve the condition because it breaks the cycle at all its pathological steps. In particular, exposure of the joint to PEMF provides that joint with external energy via the introduced pulsating uni-polar magnetic field, with a subsequent increase in the secretion of synovial fluid, all of which facilitates joint movement with less pain. The introduced pulsating uni-polar magnetic field also helps in increasing the blood supply to the muscles surrounding the joint, thereby reducing joint instability thus protecting the articular cartilage. Recent studies show that PEMF is able to stimulate chondrocyte to help in repairing of damaged cartilage. The subject invention can produce similar, if not better, results. To that end the apparatus of this invention can be positioned and oriented so that a portion of its treatment path 36 is located over the region of interest, e.g., the joint between the tibia and the femur. The ON/OFF switch can then be depressed to rotate the magnets 24A and 24B and thus produce the pulsed concentrated uni-polar electromagnetic field and direct it to that joint.

The apparatus of this invention can also be used for treating coccydynia. Coccydynia is disease which is common in women after giving birth and results in chronic pain at the lower end of the vertebral column. It is considered to be due to injury of sacro-coccygeal ligament during delivery. It also can occur in males, but such occurrences are rare. Coccydynia is resistant to all methods of treatment even surgical excision may not be helpful. Treatment with the apparatus of this invention can result in rapid healing in many cases due to its higher capacity of penetration to supply energy which is essential for the reconstruction of the healing process. To that end, the apparatus can be positioned and oriented so that the concentrated uni-polar pulsed electromagnetic field is directed to the sacro-coccygeal ligament at the sacro-coccygeal joint so that it can pass through the intervening anatomic structures.

As should be appreciated by those skilled in the art from the foregoing, the apparatus/methods of this invention produce various mechanisms for healing. In this regard, many diseases especially those that occur as a result of aging process are due to lack of local energy production. Energy is utilized by the body's tissues in many biological processes, e.g., synthesis of protein, cell division, mechanical contraction, etc. Perhaps, the most important biological process is the so-called “sodium/potassium pump mechanism”. This mechanism consumes about ⅔ of all energy of the cells and is characterised by pumping sodium out of the cell and pumping potassium in. The lack of local energy production, due to any cause, results in the cells reducing the sodium/potassium pump mechanism, whereupon there is an accumulation of sodium in the cell and the egress of potassium out of the cell. As a result, the cell becomes sick and dysfunctional (degenerated). In fact, on long standing and/or an increase in the severity of this condition, the cell dies. This process differs according to the affected site. For example, in case of the brain, this effect can present itself as Parkinson disease, Alzheimer, or dementia, according to the affected site of the brain. In case of the heart, it can be presented as heart failure, or cardiomyopathy. In other parts of the body, this effect can be presented by muscle wasting, general weakness, osteoporosis, arthritic changes of the joints and so on.

Normally, the cell membrane of healthy human cell has a negative charge of −70 mV. Diseased cells exhibit a reduction of this negative charge to be less than −70 mV, and in fact may be significantly less, e.g., start to be positive, with the lower the negativity resulting in increased disease process.

A recent study indicates that collision of pulsed magnetic field lines with body tissues generates a negative charge of −90 mV on the cell membrane of the affected cells. This is considerably more than the normally required value −70 mV. Accordingly, the application of such pulsed magnetic field can reverse the disease process of degenerated cells by neutralizing the positive or less negative cell membrane. Clinically, this means ⅔ of the energy generated by the cells can be saved and directed towards other biological process, increasing the performance of the cells.

It has also been recognized that healing of diseases can be achieved by increasing the negativity of the cell membrane through collision with magnetic field lines. Saving energy lost in sodium/potassium pump can achieve that end. In particular, saved sodium/potassium pump energy can form a surplus that can be directed to the well-being of the cells by producing more proteins and regulated cell division. For example, in case of bone, such action can increase bone mass, thereby providing a viable mechanism for treating osteoporosis. In case of muscles, excess protein formation and regulated cell division results in preventing muscle wasting. In case of cartilage, excess protein formation means repair of the defective cartilage, which is manifested clinically as healing of painful joints.

Recent studies show that stem cells are dormant and are present around deep blood vessels. Because they are dormant, the negative charge of their cell membrane is only −30 mV to −40 mV. Their exposure to a pulsed magnetic field, such as produced by the subject invention, can generate energy that capable of increasing the negativity of their cell membrane to −90 mV which can lead to their activation. Once the stem cells become active, they are suitable for repairing damaged tissues.

Some auto-immune diseases exhibit a 70% improvement by exposure to a pulsed magnetic field. The pathogenesis of auto-immune disease is precipitation of some protein particles on the cell membrane of the cells. The immune system starts to attack such tissue debris, resulting in some damage of the adjacent cell membrane and thus some cells die. It is well known that the protein has a negative charge and the cell membrane also has a negative charge, so that they repel each other. A reduction of the negativity of the cell membrane, as in case of cell sickness, facilitates the attachment of the protein particles to the cell membrane and consequently results in the auto-attack of these cells by the body's immune system. Exposure of these cells to the pulsating uni-polar magnetic field of this invention raises the negativity of the cell membrane to −90 mV, which further expels the protein particles away from the cell membrane. Thus, if the immune system attacks these free protein particles, the attack is remote from the cells so that they are not damaged, thereby resulting in an improvement of auto-immune disease.

The condition of cancer patients may also be improved by the subject invention. In this regard, the pathogenesis of cancer formation is that each cell of human body, except red blood cells (RBCs) has certain individual specific code known as human leucocytic antigen (HLA). Once any cell starts to be cancerous, the code on the cell membrane will be changed. This enables the immune system to destroy such a cancerous cell in its early stages. Recent studies shows that cell sickness associated with most cancers reduce the negativity of the cell membrane favoring the precipitation of free proteins on the cell membrane. This protein debris covers the HLA code and hides it from the immune system. Therefore, the cancer can flourish. Exposure of these cells to uni-polar pulsed, e.g., rotating, magnetic field, such as produced by the subject invention, will increase the negativity of the cell membrane to −90 mV, whereupon the free protein is expelled away from the cell membrane. Thus the HLA code is again exposed to the immune system, to enable the immune system to attack the cancer cell to destroy it.

It should be pointed out at this juncture that various other diseases/conditions can be treated with the apparatus and methods of this invention. In particular, it is envisioned that the subject invention can be used for treatment of all diseases that respond to the prior PEMF apparatus and methodologies. Thus, as described above the subject invention can be used for treatment of Alzheimer, Parkinson's disease, dementia, and stroke (vascular insult of the brain). The subject invention can be used for card iomyopathy, heart failure, coronary artery diseases (CAD) which have a common problem, i.e., a lack of local energy by narrowing of the artery. Activation of stem cells inside the fibrous tissues by increasing the negativity of the cell membrane will replace this fibrous tissue of the heart into healthy heart muscles. In the case of the musculo-skeletal system, the subject invention can be used to treat osteoporosis, joint arthritis, and muscle wasting that causes weakness and debility in old age. The foregoing examples of diseases/conditions are merely exemplary of various diseases/conditions that can be effectively treated by the subject invention.

Like the apparatus and methods of our copending application PCT Application PCT/IB2013/002441, the apparatus and methods of the subject invention offers numerous advantages over the conventional electromagnetic-coil based apparatus/methods for providing PEMF. Yet, the subject invention does so without resulting in the production of unwanted electromagnetic-coil produced heat, which limits the amount of time the apparatus can be used. Moreover, the subject invention, like the apparatus and methods of our copending application PCT Application PCT/IB2013/002441, by-passes the skin barrier and allows penetration to deeper tissues/structures, e.g., three inches or greater. In fact apparatus constructed in accordance with this invention and the methods of this invention apply a pulsed uni-polar magnetic field produced by one or more permanent magnets at virtually any depth within the body of the patient to apply maximum energy to the region of interest. Moreover, the narrow or somewhat collimated field produced by the apparatus of this invention results in the delivery of the pulsed uni-polar magnetic field to a reduced size surface or volume so that it can absorb a great part of applied energy, as opposed to prior art PEMF systems which are characterised by dissipated or widely divergent fields. All of this is accomplished by use of at least one very strong permanent magnet, e.g., Neodymium N52 magnet, in lieu of electromagnets to produce the magnetic field. In particular, with the at least one strong permanent magnet being rotated or otherwise periodically moved in a path over the region of interest in the patient it produces a pulsed field. Accordingly, the strong magnetic field produced by the magnet(s) periodically penetrate to deep tissues with a sufficient amount of energy to reproduce the effects of prior art PEMF therapy achieved through use of electromagnets, yet without concomitant heat production resulting from the use of electromagnets. Therefore, the apparatus of the subject invention can be utilized for long periods of time without need to shut down the apparatus to cool it.

Moreover, like the apparatus of our copending application PCT Application PCT/IB2013/002441, the apparatus of this invention is constructed to that the field produced is effectively uni-polar. Since it is recognized that the northern pole is more physiologically beneficial than southern pole, the exemplary preferred embodiments of this invention are constructed so that the northern pole will be directed to the diseased part of human tissues (the region of interest) while the southern pole will be hidden or isolated by surrounding it with the Mu-metal shielding.

In closing it should be noted that the particular components and their arrangements as discussed above and as shown in the drawings are merely exemplary. Thus, other components can be used in lieu of those disclosed. By way of example, and not limitation, the shield 26 may be formed of some material having similar magnetic field modifying effects as Mu-metal. Moreover, the apparatus may make use of any number of permanent magnets. Thus, it can use a single permanent magnet which is rotated through the path 36, or may make use of three or more permanent magnets which are rotated through the path. The apparatus can be of any suitable size and is preferably sufficiently small that it can be hand-held by a user, but can if desired be supported by some mount, e.g., an adjustable member.

Without further elaboration the foregoing will so fully illustrate our invention that others may, by applying current or future knowledge, adopt the same for use under various conditions of service.

I claim:

1. Apparatus for applying pulsed uni-polar magnetic energy to an anatomic region of interest located within the body of a patient, said apparatus comprising at least one permanent magnet, a body of Mu-metal shielding, and a motor, said at least one permanent magnet producing a magnetic field having a longitudinal axis, a first pole of a first polarity and a second pole of a second polarity, with said longitudinal axis extending through said poles and with lines of flux of said magnetic field extending outward from said first pole generally parallel to said longitudinal axis, said at least one permanent magnet being located within said housing and being surrounded by said Mu-metal shielding except for said first pole of said at least one permanent magnet, whereupon said first pole is exposed and from which lines of flux of said magnetic field emanate, said at least one magnet being coupled to said motor, said motor being arranged to be operated to repeatedly move said at least one magnet through a predetermined path, whereupon a portion of that path can be disposed adjacent the anatomic region of interest of the patient to cause said exposed pole of said at least one permanent magnet to periodically direct said lines of flux to the region of interest in the patient.

2. The apparatus of claim 1 wherein said apparatus comprises two permanent magnets, said magnets being mounted on a member spaced diametrically apart from each other, said member having a central rotation axis about which said member is rotated to move said two permanent magnets in a circular path extending about said central rotation axis.

3. The apparatus of claim 1 wherein said motor is mounted within said housing.

4. The apparatus of claim 1 wherein said body of the metal shielding includes a window through which said lines of flux from said first pole of said at least one magnet can emanate.

5. The apparatus of claim 3 wherein said motor is an electric motor.

6. The apparatus of claim 1 wherein said first pole is the North pole of said at least one permanent magnet.

7. The apparatus of claim 2 wherein said first pole is the North pole of each of said two permanent magnets.

8. The apparatus of claim 1 wherein said at least one permanent magnet is a super-magnet material.

9. The apparatus of claim 8 wherein said super-magnet material comprises Neodymium.

10. The apparatus of claim 9 wherein said super-magnet material comprises Neodymium N52.

11. The apparatus of claim 1 wherein said apparatus is located within a housing.

12. The apparatus of claim 11 wherein said housing is arranged to be manually held by a user of the apparatus.

13. The apparatus of claim 1 wherein said motor is operated to periodically direct said lines of flux to the region of interest in the patient at an on-off pulse rate of approximately 25-50 per second.

14. The apparatus of claim 13 wherein said pulse rate comprises at least approximately 30-40 percent on and approximately 70-60 percent off.

15. A method of treating medical condition of a patient by applying a pulsed uni-polar magnetic field to an anatomic region of interest in the body of the patient, said method comprising:
a) providing apparatus comprising at least one permanent magnet, a body of Mu-metal shielding, and a motor, said at least one permanent magnet producing a magnetic field having a longitudinal axis, a first pole of a first polarity and a second pole of a second polarity, with said longitudinal axis extending through said poles and with lines of flux of said magnetic field extending outward from said first pole generally parallel to said longitudinal axis, said at least one permanent magnet being located within said housing and being surrounded by said body of Mu-metal shielding except for said first pole of said at least one permanent magnet, whereupon said first pole is exposed and from which lines of flux of said magnetic field emanate;
b) disposing said housing adjacent the region of interest in the body of the patient; and
c) operating said motor to repeatedly move said at least one magnet through a predetermined path, whereupon a portion of said path is disposed aligned with the anatomic region of interest of the patient to cause said exposed pole of said at least one permanent magnet to periodically direct said lines of flux to the region of interest in the patient each time said at least one magnet reaches said portion of said path.

16. The method of claim 15 wherein said motor is operated to periodically direct said lines of flux to the region of interest in the patient at an on-off pulse rate of 25-50 per second.

17. The method of claim 16 wherein said pulse rate comprises at least approximately 30-40 percent on.

18. The method of claim 15 wherein said medical condition comprises brain pathology, and wherein the region of interest is in the brain of the patient.

19. The method of claim 18 wherein said brain pathology is selected from the group comprising Alzheimer's disease, Parkinson's disease, dementia, migraine and senile atrophy of the brain.

20. The method of claim 15 wherein said medical condition comprises cardiac disease, and wherein the region of interest is in the heart of the patient.

21. The method of claim 20 wherein said cardiac disease is selected from the group comprising coronary artery disease, cardiomyopathy, and prior myocardial infarction.

22. The method of claim 15 wherein said medical condition comprises an orthopedic condition, and wherein the region of interest is in the bone of the patient.

23. The method of claim 22 wherein said orthopedic condition is selected from the group comprising osteoporosis, avascular necrosis, non-union or delay bone healing after fracture, osteomyelitis, intervertebral disc herniation or compression, arthritis, and coccydynia.
